(19) United States
(12) Patent Application Publication
(10) Pub. No.: US 2026/0263010 A1
(43) Pub. Date: Sep. 10, 2026

(54) SYSTEMS AND METHODS FOR REMOVING STIMULATION ARTIFACTS FROM PHYSIOLOGICAL RECORDINGS

(71) Applicant: Ohio State Innovation Foundation, Columbus, OH (US)

(72) Inventors: Jeffrey SKIDMORE, Apex, NC (US); Shuman HE, Columbus, OH (US)

(21) Appl. No.: 19/164,476

(22) PCT Filed: Mar. 15, 2024
(86) PCT No.: PCT/US2024/020175

§ 371 Date: Sep. 11, 2025

Related U.S. Application Data
(60) Provisional application No. 63/490,580, filed on Mar. 16, 2023.

(51) Int. Cl. A61B 5/00; A61B 5/388

(52) U.S. Cl. CPC A61B 5/7217; A61B 5/388

(57) ABSTRACT

Disclosed herein are of systems, methods, and computer-program products for removing stimulation artifacts of electrical physiological recordings that contain both the stimulation artifact and the neural response.

### CROSS REFERENCE TO RELATED APPLICATION

This application claims priority to and benefit of U.S. Provisional Patent Application Ser. No. 63/490,580 filed Mar. 16, 2023, which is fully incorporated by reference and made a part hereof.

### GOVERNMENT SUPPORT CLAUSE

This invention was made with government support under grant/contract number DC016038 awarded by the National Institutes of Health. The government has certain rights in the invention.

### BACKGROUND

As shown in FIG. 1, cochlear implants convert sounds (picked up by a microphone) into electrical representations (in the sound processor) which are transmitted through the head and stimulate nerves surrounding the cochlea. Cochlear implants bypass the normal hearing pathway (i.e. middle ear bones and inner ear hair cells), which is usually not functional when somebody is deaf. However, cochlear implants still rely on the cochlear nerve to be functional in order to carry the signal to the brain for further processing and interpretation (as shown in FIG. 2).

In humans, there is no way to directly evaluate how well the cochlear nerve functions. However, as shown in FIG. 3, user defined stimuli can be sent through one electrode to stimulate the surrounding neurons. The neurons then respond and the electrical response can be recorded by a neighboring electrode. The neural response is called the electrically-evoked compound action potential (eCAP). The eCAP response can be characterized as an indirect measure of neural function.

There are clinical applications of detected eCAPs. See, for example, “SYSTEMS AND METHODS FOR DETECTING THE PRESENCE OF ELECTRICALLY EVOKED COMPOUND ACTION POTENTIALS (eCAPs), ESTIMATING SURVIVAL OF AUDITORY NERVE FIBERS, AND DETERMINING EFFECTS OF ADVANCED AGE ON THE ELECTRODE-NEURON INTERFACE IN COCHLEAR IMPLANT USERS,” PCT/US2021/033604, published as WO 2021/236059 on Nov. 25, 2021, which is fully incorporated by reference and made a part hereof.

However, a challenge for any clinical application of eCAP technology is that the voltage recorded from the stimulation (i.e., stimulation artifact) is 1 to 2 orders of magnitude bigger than the voltage recorded from the neural response. Therefore, the stimulation artifact must be removed from the recording to view and isolate the neural response. Recording artifacts are also present in the eCAP measurements but are much smaller than the stimulation artifacts.

Previous attempts at resolving this challenge include the alternating polarity method, which assumes cathodic leading and anodic leading pulses result in the same neural response (amplitude and latency), which has repeatedly been shown to not be true. Other methods include forward masking methods. Single pulse forward masking assumes that all neurons are in refractory state after a first pulse. This is sometimes true but cannot be guaranteed in every instance. This method requires an expert to determine for each recording individually. Pulse train forward masking assumes the stimulation artifact from a single pulse is the same as the stimulation artifact from the last pulse of a pulse train, which is not true.

Therefore, systems and methods are desired that overcome challenges in the art, some of which are described above. More specifically, there is a need for systems and methods to reliably remove the stimulation artifact, especially for trains of stimulation pulses, from eCAP recordings, which are used in cochlear implants.

### SUMMARY

Disclosed and described herein are systems and methods to address the above-described challenges. In particular, systems and methods are disclosed to remove stimulation artifacts from physiological recordings of a stimulation and the resultant neural response. As noted above, the eCAP is a measure of the responses of auditory nerve fibers that can recorded directly from a cochlear implant. A challenge for clinical application of eCAP technology is identifying the presence of an eCAP instead of measurement noise, including stimulation artifacts and recording artifacts. The disclosed systems and methods remove the stimulation artifacts from the recorded and measured values.

Systems and methods are disclosed herein for removing stimulation artifacts from electrical recordings that contain both the stimulation artifact and the neural response. One embodiment of the method comprises recording an electrical trace (“A” trace) for a first period of time (e.g., 3200 microseconds (μs)). The stimulation artifact is modeled as a rational function. Therefore, the rational function is fit to a first portion (e.g., first few μs) of the recorded A trace, and to a second portion of the recorded A trace (the second period may be from after the first 2200 μs of the recorded A trace to the end of the A trace), wherein there is no neural response during the second portion (e.g., after 2200 μs) and the stimulation artifact reaches a linear asymptote. The modeled rational function is then subtracted from the recorded A trace, leaving only the neural response (i.e., the eCAP). In some instances, the rational function is a hyperbola function. Systems for implementing this method are also described herein.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems.

FIG. 1 is an image illustrating an example of a cochlear implant.

FIG. 2 is an image illustrating how cochlear implants still rely on the cochlear nerve to be functional in order to carry the signal to the brain for further processing and interpretation.

FIG. 3 is an illustration of how user defined stimuli can be sent through one electrode of a cochlear implant to stimulate the surrounding neurons, with the compound neural response (i.e., eCAP) being recorded by a different electrode of the cochlear implant.

FIGS. 4A-4C illustrate examples of conventional methods of removing stimulation artifacts from recorded eCAP traces.

FIG. 5 is a flowchart that discloses an exemplary method to remove stimulation artifacts from physiological recordings.

FIGS. 6A and 6B illustrate that a rational function (e.g., hyperbola) best characterizes stimulation and recording artifacts (i.e., B−C+D trace of a fully masked stimulation).

FIGS. 7A and 7B illustrate that for the various A traces, with varying current stimulation levels as shown in FIG. 7A, the B−C+D trace of a fully masked stimulation for each of the A traces in FIG. 7A, as shown in FIG. 7B, can be modeled as a hyperbola, which is a specific form of a rational function.

FIGS. 8A-8E provide additional details regarding the method described in relation to FIG. 5, above.

FIGS. 9A-9B illustrate that the disclosed methods can be applied to pulse trains as well as to single-pulse situations.

FIGS. 10A-10H illustrate comparisons of determining the neural response (i.e., eCAP) using the conventional forward masking method, described herein, as compared to the disclosed methods where FIGS. 10A-10D illustrate examples of removing stimulation artifacts from physiological recordings after a single pulse, and FIGS. 10E-10H illustrate examples of removing stimulation artifacts from physiological recordings after multiple pulses (a series of single pulses or a pulse train).

FIGS. 11A-11E illustrate an application of the disclosed methods to obtain neural recording of speech stimuli in cochlear implant patients (i.e., electrical instead of acoustic hearing).

FIG. 12 illustrates an exemplary computing device that can be used according to embodiments described herein.

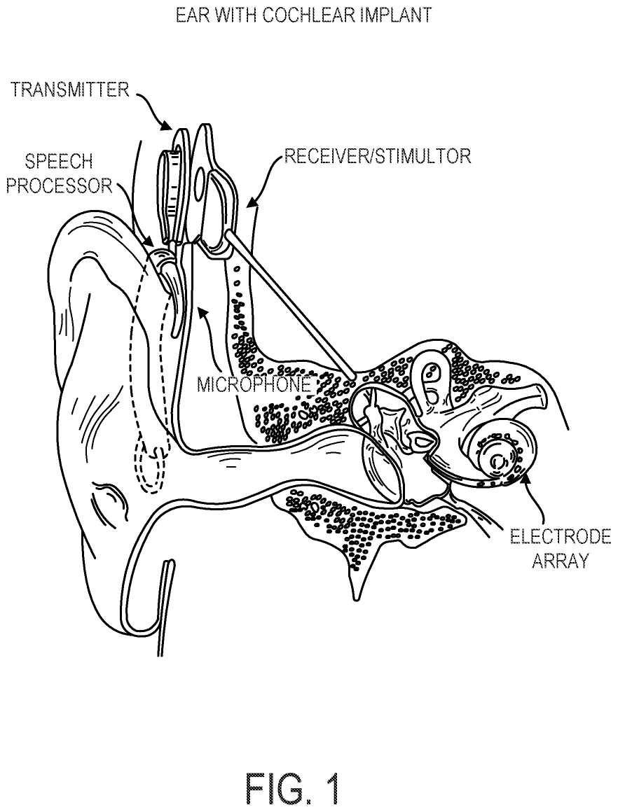

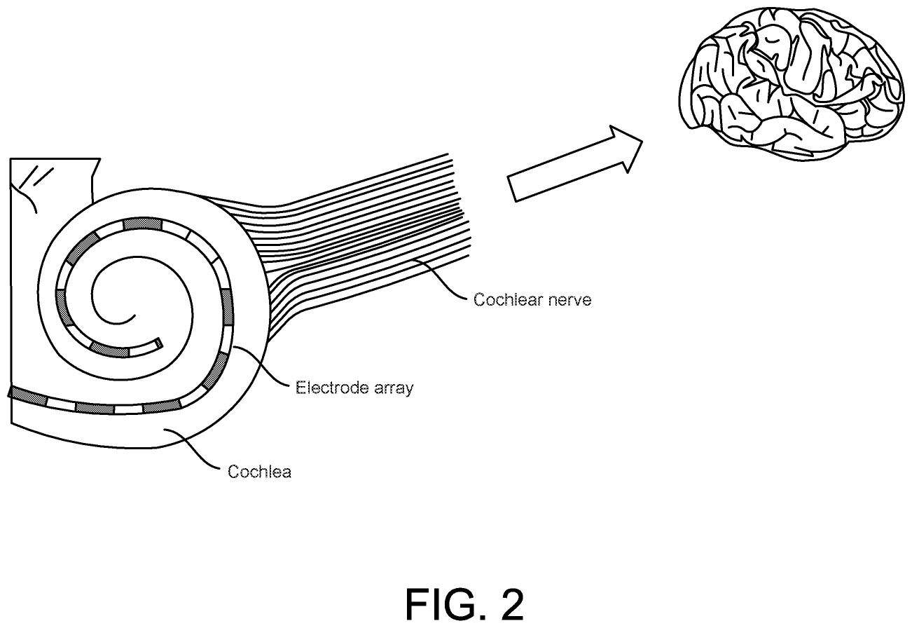

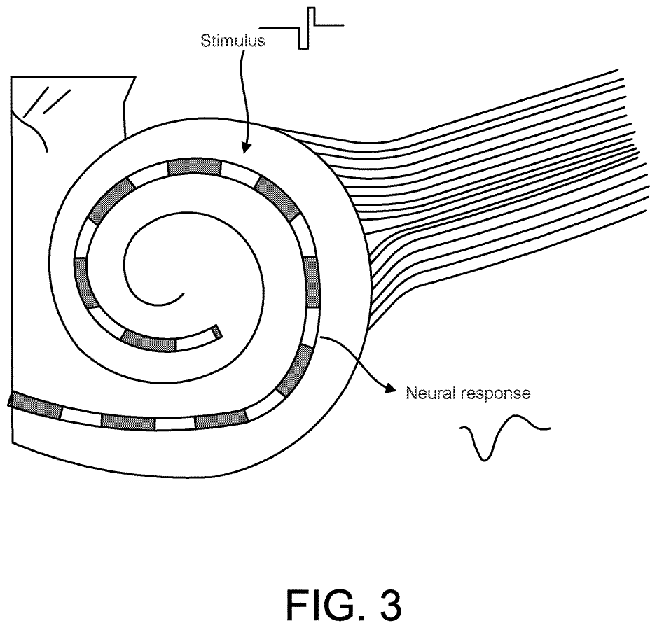

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

The present methods and systems may be understood more readily by reference to the following detailed description of preferred embodiments and the Examples included therein and to the Figures and their previous and following description.

What is desired is systems and methods to reliably remove the stimulation artifact, especially for trains of stimulation pulses, from eCAP recordings, which are used in cochlear implants.

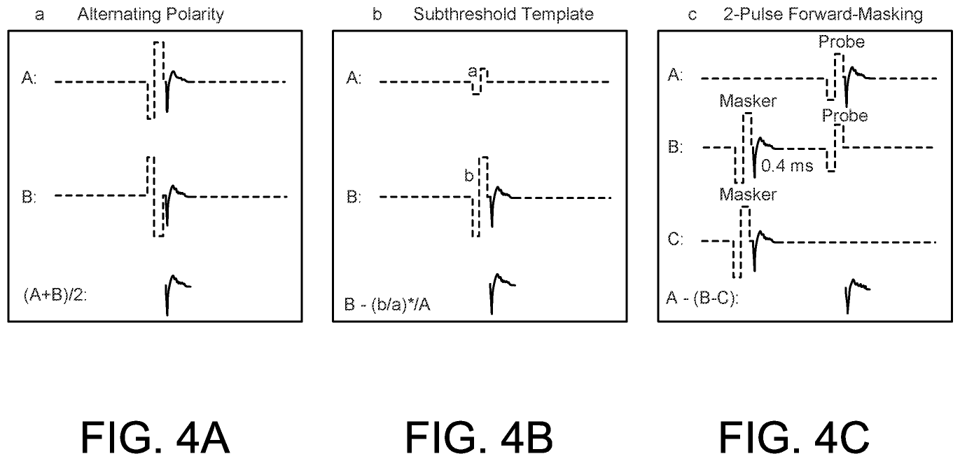

FIGS. 4A-4C illustrate examples of conventional methods of removing stimulation artifacts from recorded physiological traces. All of these conventional methods have limitations that make it challenging to use eCAP traces in clinical environments. FIG. 4A illustrates the alternating polarity method. In this method, a bi-phasic pulse is presented in two configurations—cathodic-leading (see trace “A” of FIG. 4A), and anodic-leading (see trace “B” of FIG. 4A). The theory behind this method is that the stimulation artifact will be removed when averaging the voltage recordings of these two simulations. However, this method relies on the assumptions that the stimulation artifacts from the two records are symmetric (i.e., cancel each other out) and that the eCAP response is the same for both stimulations. Both assumptions have repeatedly been shown to not be valid. FIG. 4B illustrates the subthreshold template method. In this method, a template of the stimulation artifact is obtained by measuring the voltage of a sub-threshold stimulation (see trace “A” of FIG. 4B) that does not produce a neural response. The voltage is then measured for the desired stimulation pulse at a higher stimulation level (see trace “B” of FIG. 4B). The subthreshold template is then scaled according to the difference in stimulation level and subtracted from the primary recording (see trace “B” of FIG. 4B). However, this method relies on the assumption that the stimulation artifact scales linearly with stimulation level which has repeatedly been shown to not be a valid assumption. And FIG. 4C illustrates the 2-pulse forward masking method. Forward masking (FIG. 4C) is the most used conventional method. The idea is to create a “template” (i.e. voltage trace) that represents the stimulation artifact. This is done, in theory, by using a masker pulse that puts the neurons in a refractory state, so that there is not a neural response to the probe pulse (see trace “B” of FIG. 4C). By subtracting of the recording of the masker alone (see trace “C” of FIG. 4C), the artifact “template” is created. This template (B trace−C trace) can be subtracted from the stimulation trace of interest (see trace “A” of FIG. 4C), presumably leaving just the neural response (i.e., eCAP). However, this method relies on the assumption that the masker fully puts all neurons in a refractory state. This assumption is only valid for some stimulation conditions.

Note: A “D” trace represents the recording artifact, but because it is much smaller than the stimulation artifact it cannot be shown on the scale of FIGS. 4A-4C, but ought to be considered as well. Therefore, D is included in the analyses that follow.

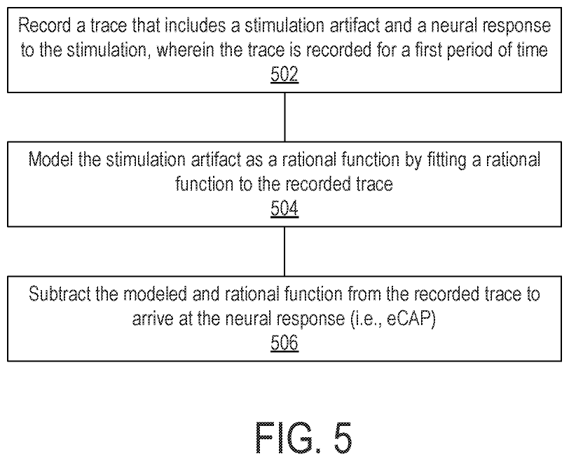

FIG. 5 is a flowchart that discloses an exemplary method to remove stimulation artifacts from physiological recordings. As shown in FIG. 5, the process begins at 502 by recording an electrical trace (this may be referred to as an, “A” trace in the specification, claims and figures, herein) that included both the stimulation artifact and a resultant neural response. The A trace is recorded for a first period of time (e.g., for about 3200 microseconds (μs)).

At step 504, the stimulation artifact is modeled as a specific order rational function by fitting a rational function to the recorded trace. For example, the stimulation artifact may be modeled as a hyperbola, which is fit to the A trace. In some instances, the rational function is fit to a first portion of the A trace and a second portion of the recorded A trace. For example, the rational function (e.g., hyperbola) is fit to only a first few μs of data from the recorded A trace, and data from the recorded A trace after approximately 2200 μs. Generally, the first few μs of data is a range of about 20 μs. For example, the first few μs of data may start at about 179 μs of the recorded A trace and go to about 199 μs. It can start sooner (e.g. 149 μs) and be a longer range (to about 100 μs). This is because a neural response, if any, at the beginning of A trace recording window is tiny compared to the stimulation artifact during that first portion of the recorded A trace. Further, there is typically no neural response during the second portion of the recorded A trace (e.g., after about 2200 μs) and the stimulation artifact reaches a linear asymptote. At step 506, the modeled and fit rational function (e.g., the hyperbola) is subtracted from the recorded A trace to get just the neural response (eCAP).

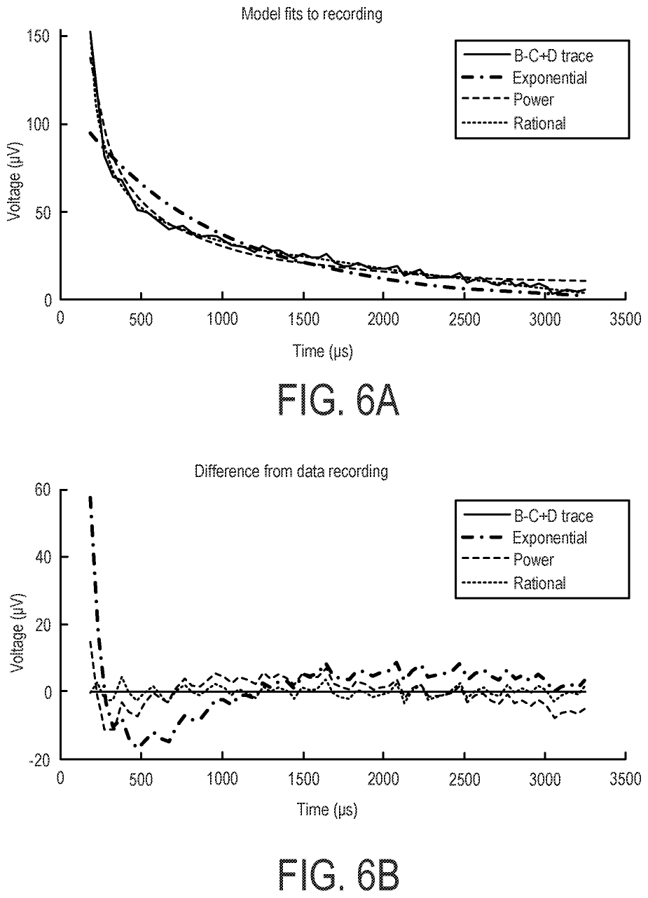

This process is shown in greater detail with reference to FIGS. 6A and 6B, which illustrate that a hyperbola (i.e., rational function) best characterizes the artifacts (i.e., B−C+D trace of a fully masked stimulation). FIG. 6A illustrates the B−C+D trace of the fully masked stimulation and how an exponential function (i.e., y=ae^{bx}+ce^{dx}), a power function (i.e., y=ax^{b}+c) and the disclosed rational function fit to the B−C+D trace. The rational function in this instance is a hyperbola, which can be described by the following rational function:

$$y = \frac{{p}_{1} {x}^{2} + {p}_{2} x + {p}_{3}}{x + {q}_{1}}$$

It has the following asymptotes that match the recorded data:
Vertical asymptote: x=−q₁
Slanted asymptote: y=p₁x+P₂−p₁q₁

FIG. 6B illustrates the difference from the data recording for each of the exponential, power and rational functions as compared to a 0 line (no difference). As can be seen, the rational function tracks the recorded data much closer than either the exponential or power functions.

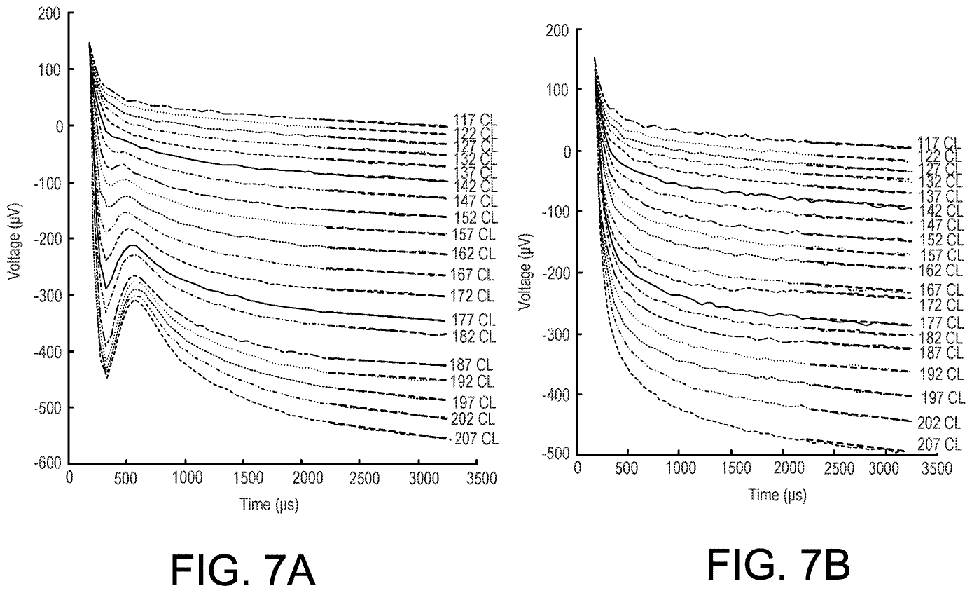

FIGS. 7A and 7B show that for the various A traces, with varying current stimulation levels as shown in FIG. 7A, the B−C+D trace of a fully masked stimulation for each of the A traces in FIG. 7A, as shown in FIG. 7B, can be modeled as a hyperbola, which is described by the rational function above. As can be seen in FIG. 7A, the A traces contain a visible neural response from about 300 us to 800 us at the higher stimulation levels, as well as the stimulation artifact throughout the entire recording. Each B−C+D trace (having been fully masked) of FIG. 7B shows a decaying artifact, significant non-zero slope, and normal residuals of “tail.” This demonstrates clearly that the stimulation artifact can be modeled as a hyperbola at all of the stimulation levels.

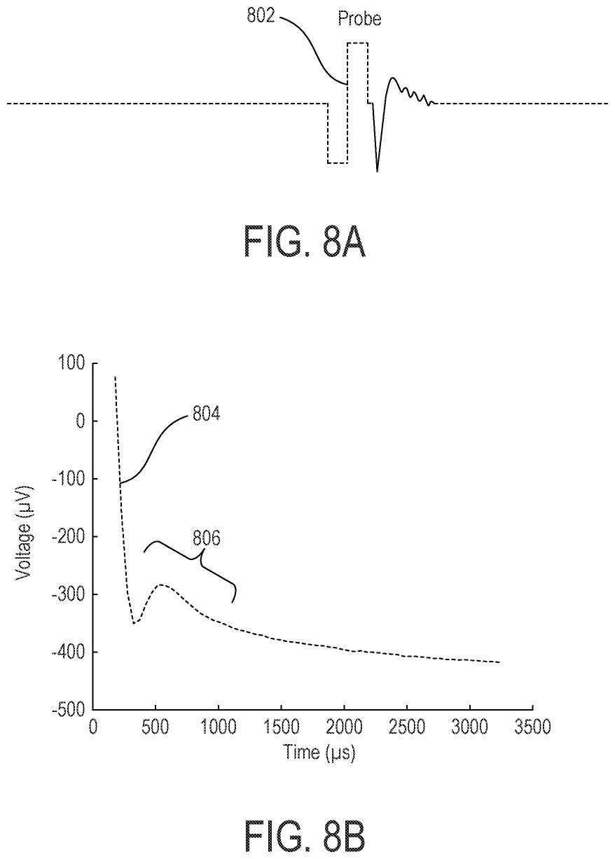

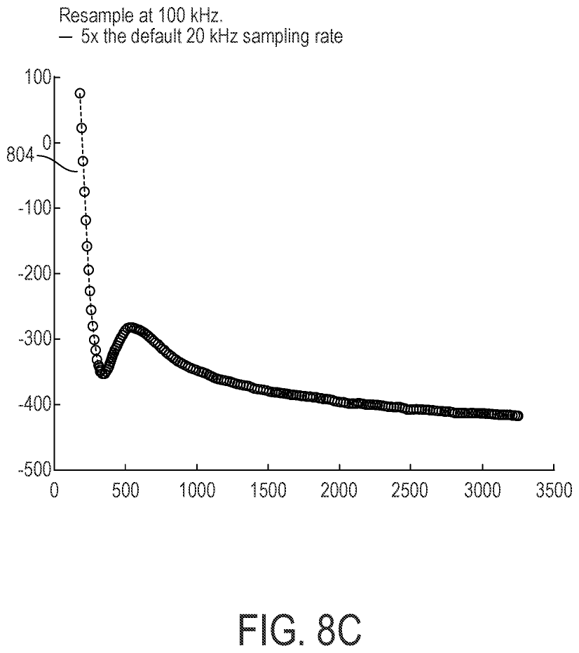

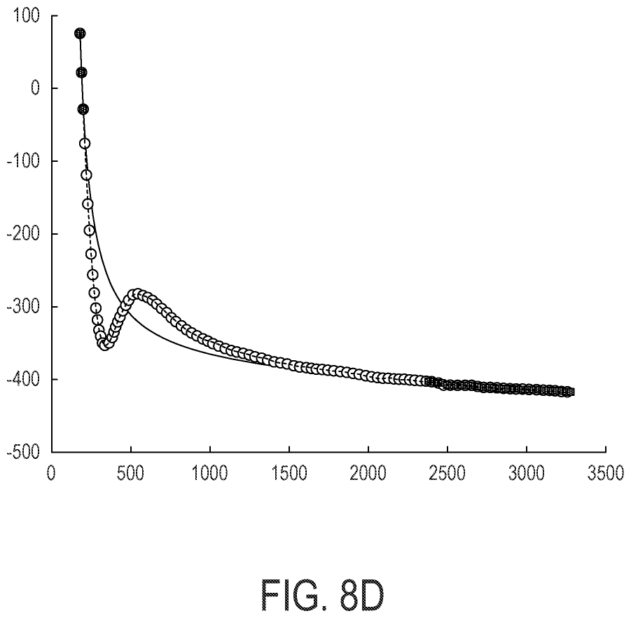

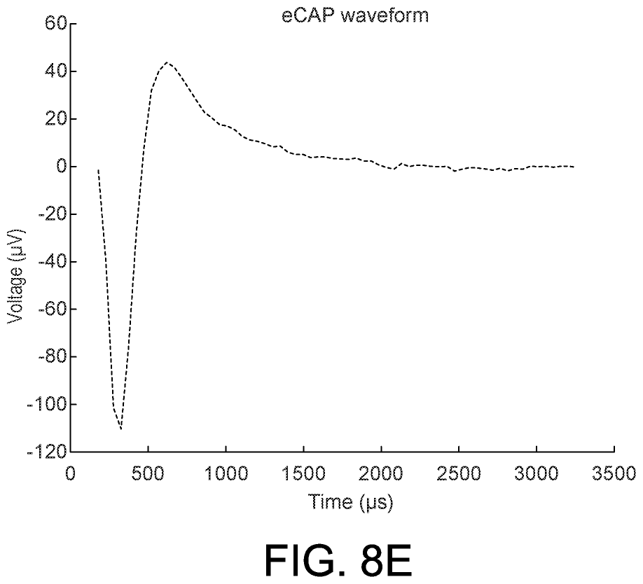

The following figures (FIGS. 8A-8E) provide details regarding the method described in relation to FIG. 5, above. FIG. 8A illustrates a probe pulse stimulation 802 and FIG. 8B shows the resultant trace (“A” trace) 804, which contains the stimulation artifact 806, and the neural response. As shown in FIG. 8B, the recording period is approximately 3200 μs. As illustrated in FIG. 8C, the A trace 804 is resampled at a rate that is greater than its original sampling rate. For example, the A trace 804 may have been originally sampled at 20 kHz, but is re-sampled at 100 kHz. FIG. 8D illustrates that a rational function (e.g., a hyperbola) is fit to some points of the up-sampled waveform. For example, in FIG. 8D the rational function is fit to three points (e.g., 179, 189, and 199 us) during a first portion of the waveform where the neural response (if present) is much smaller than the stimulation artifact. The rational function is then fit to points on the waveform that occur in a second portion of the waveform (e.g., data points after 2200 us), which contain no neural response at these time points (only stimulation artifact). In some instances, an additional point selected at a point between the min and max peaks in the combined waveform (not required but helpful in some cases) is used to fit the rational function to the waveform. FIG. 8E illustrates the waveform of just the neural response as the modeled stimulation artifact (e.g., hyperbola) has been mathematically subtracted from the original record (“A” trace—that include both the stimulation artifact and the neural response) to get just the neural response (i.e., eCAP).

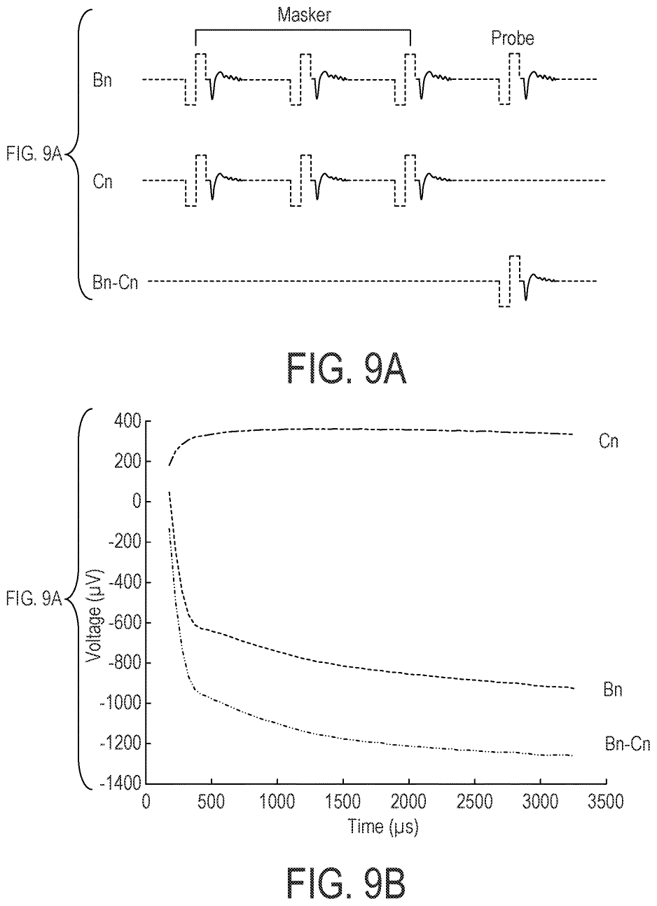

FIGS. 9A and 9B illustrate that the above-described method can be applied to pulse trains. As shown in FIG. 9A, recorded trace Bₙ includes a series of masker pulses prior to application of a probe pulse. Recorded trace Cₙ includes the masker artifact and neural response responsive to the pulses of trace Bₙ. Recorded trace Cn is subtracted from recorded trace Bₙ in order to get just the probe pulse stimulation artifact and neural response (Bₙ-Cₙ), and then all steps for a single pulse, as described herein, are followed. This is graphically shown in FIG. 9B. The rational function is fit to the resultant Bₙ-Cₙ waveform, and then subtracted from the Bₙ-Cₙ waveform to arrive at the neural response after a series of pulses. Advantages of being able to determine the neural response after a series of pulse include identifying functional neural response properties of the auditory nerve (e.g., neural adaptation, neural adaptation recovery, sensitivity to amplitude modulation cues) and understanding neural encoding of speech stimuli.

### A. Examples of Removing Stimulation Artifacts from Physiological Recordings after Single and Multi-Pulses

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process.

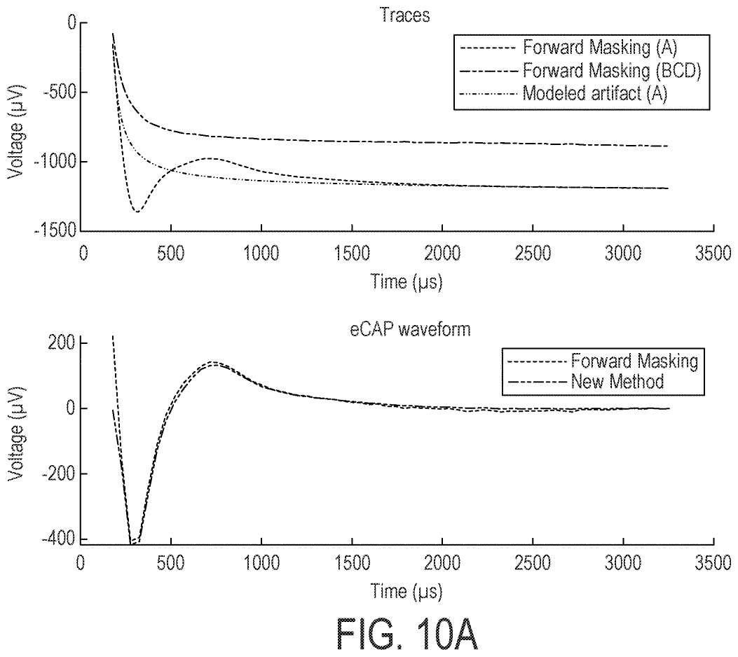

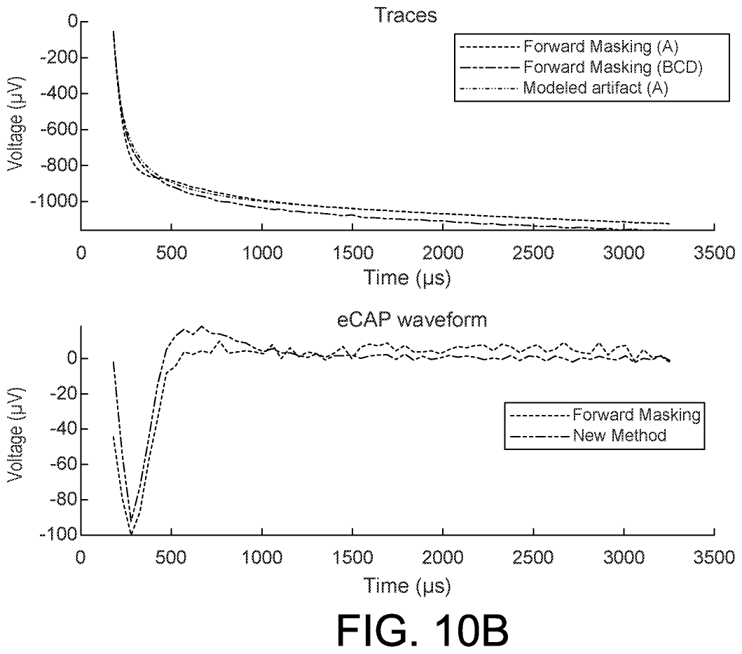

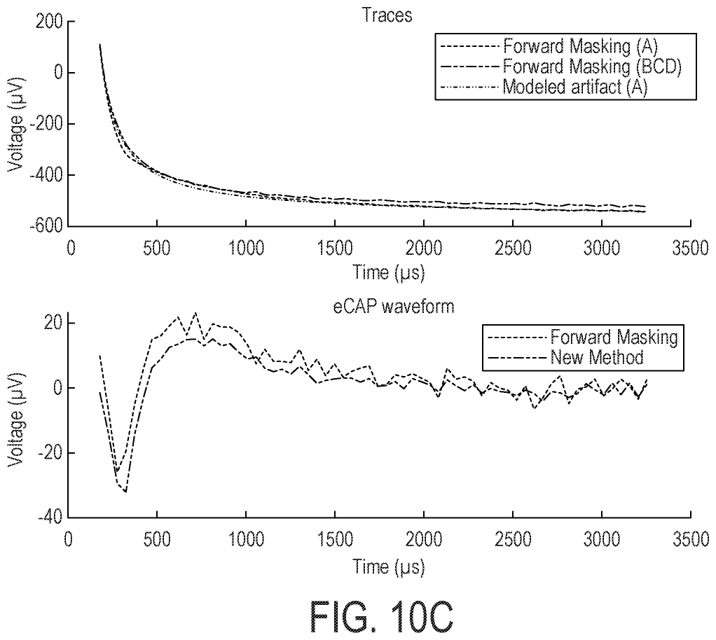

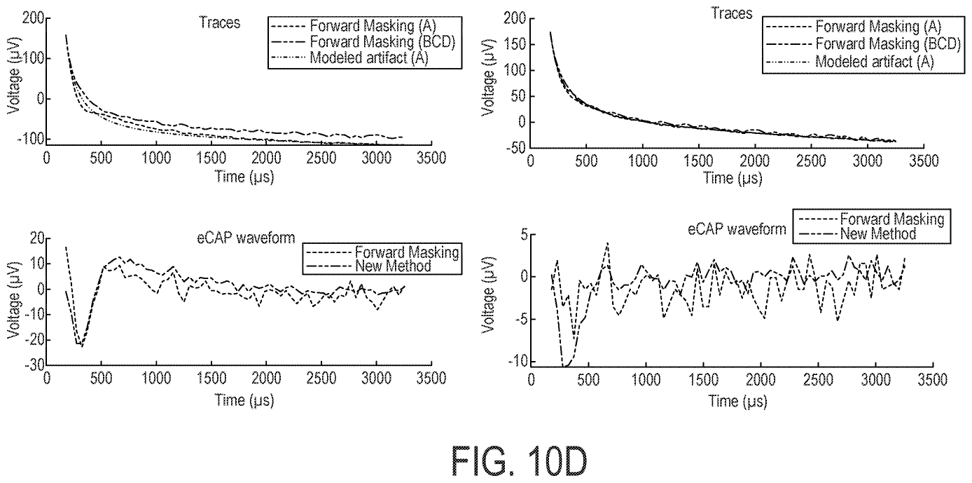

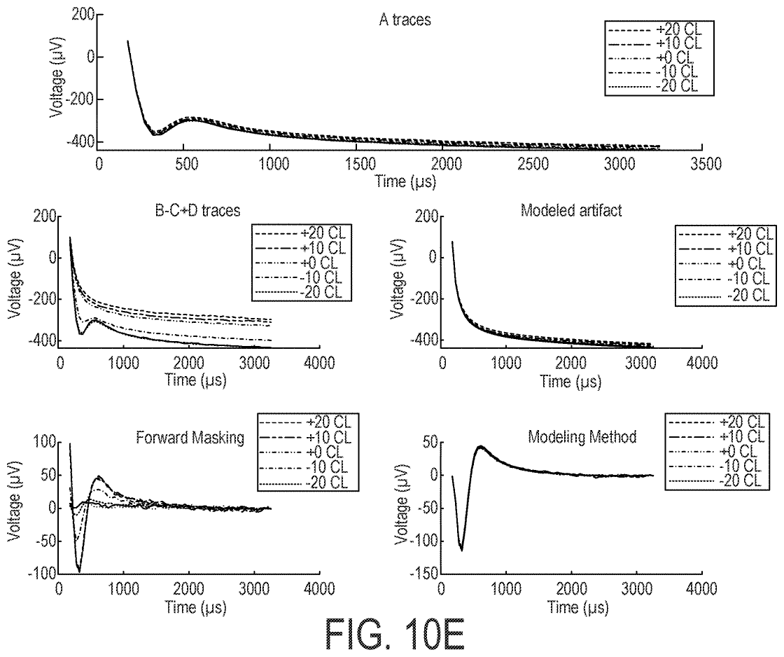

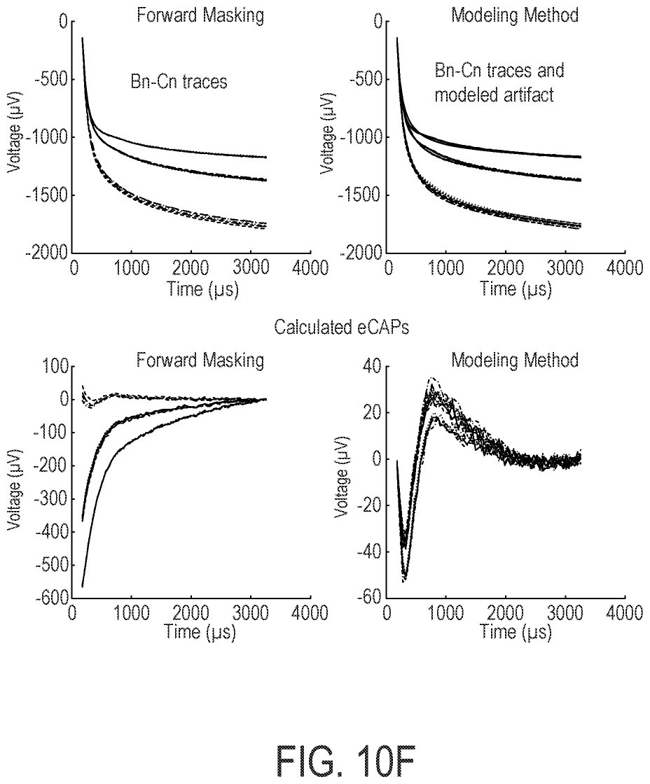

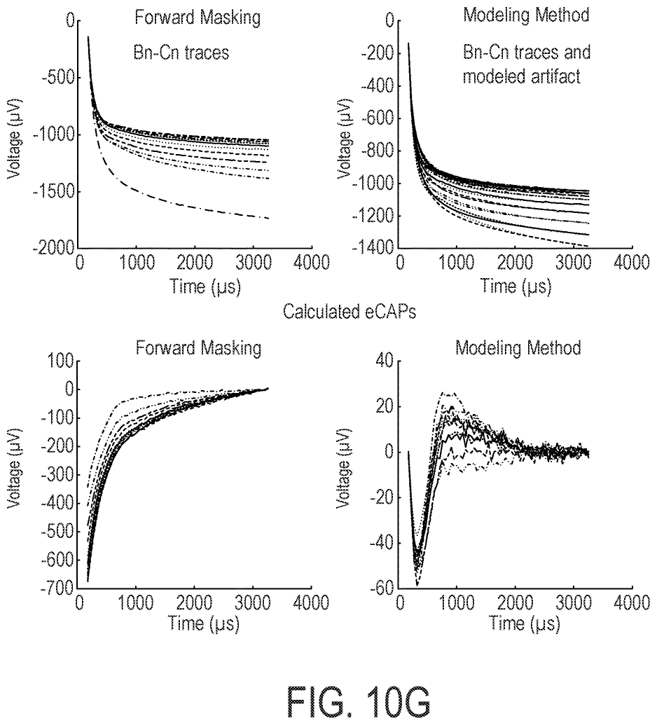

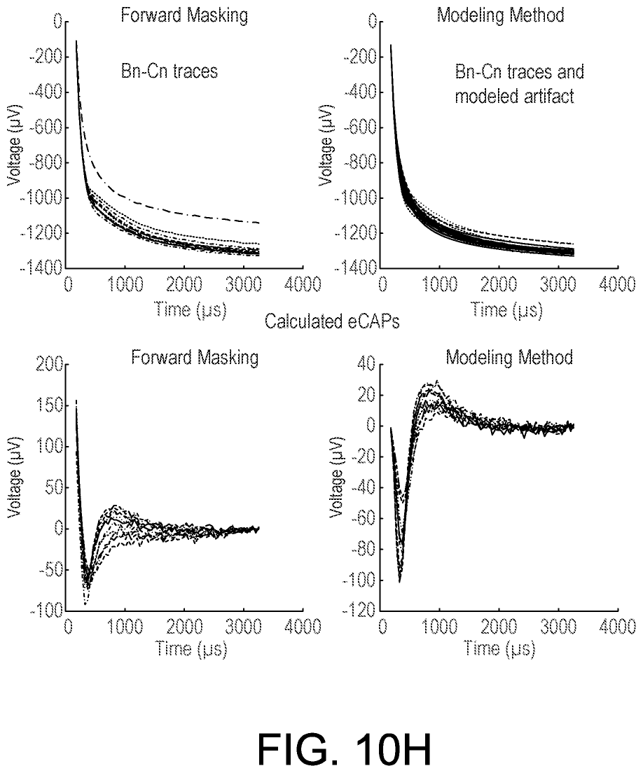

FIGS. 10A-10H illustrate comparisons of determining the neural response (i.e., eCAP) using the conventional forward masking method, described herein, as compared to the disclosed methods. FIGS. 10A-10D illustrate examples of removing stimulation artifacts from physiological recordings after a single pulse, and FIGS. 10E-10H illustrate examples of removing stimulation artifacts from physiological recordings after multiple pulses (a series of single pulses or a pulse train). In each instance of multiple pulses, conventional forward masking fails.

### B. Neural Recording of Speech Stimuli in Cochlear Implant Patients (i.e., Electrical Instead of Acoustic Hearing)

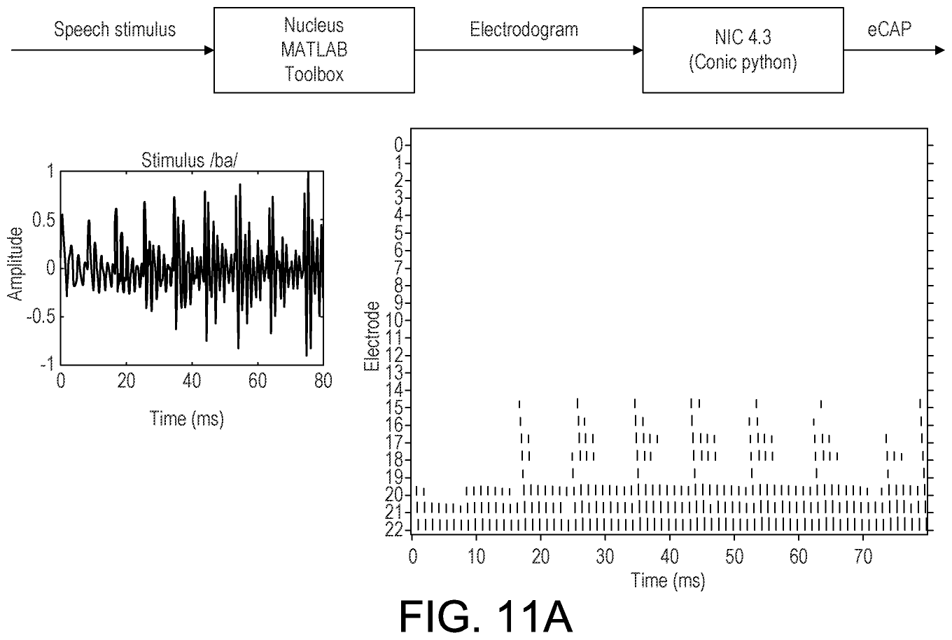

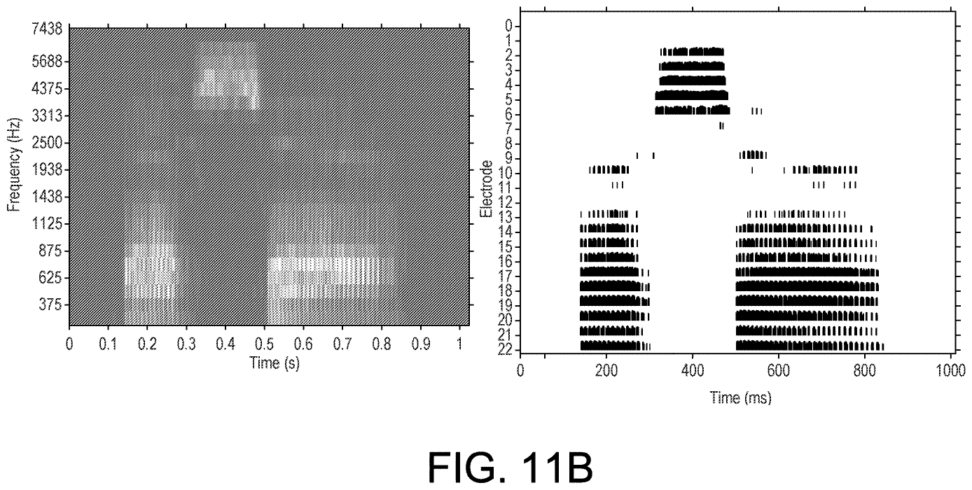

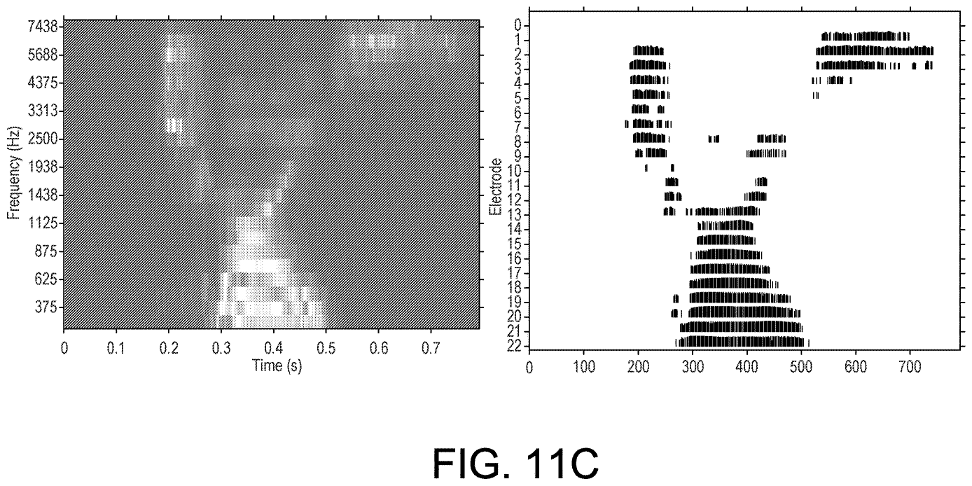

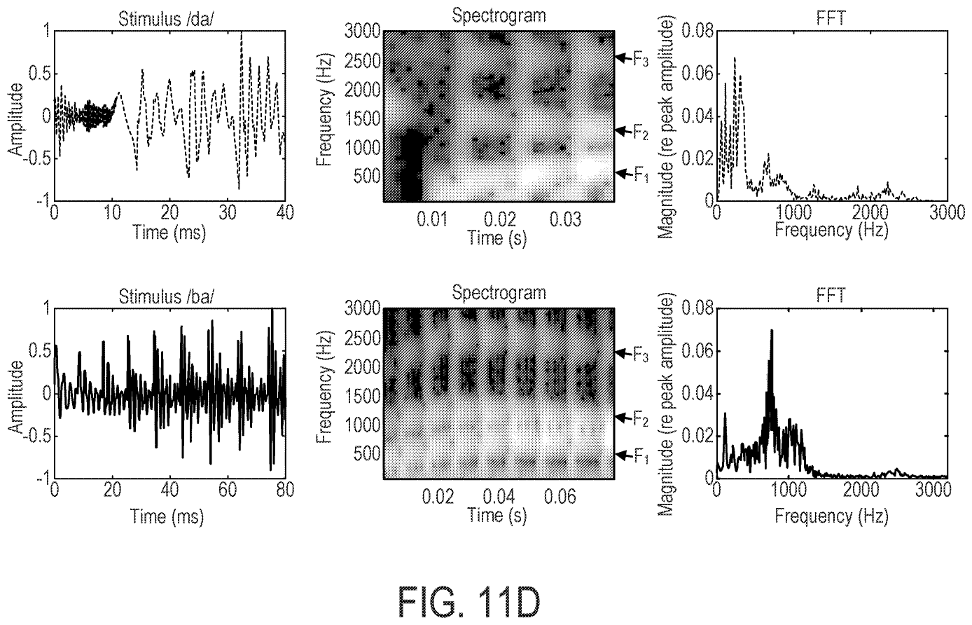

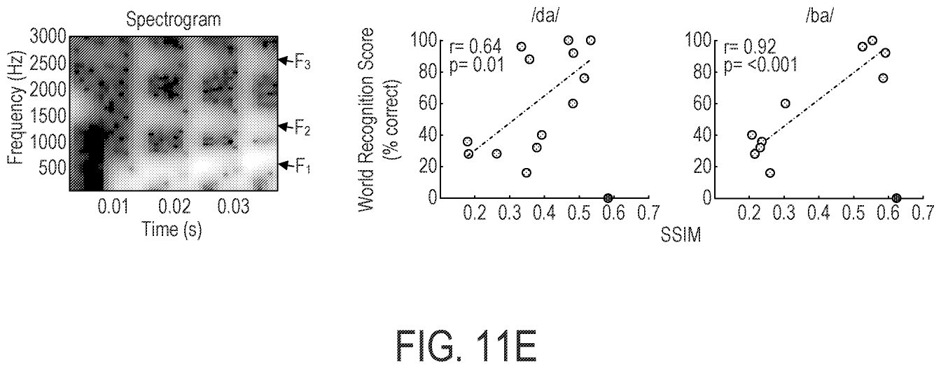

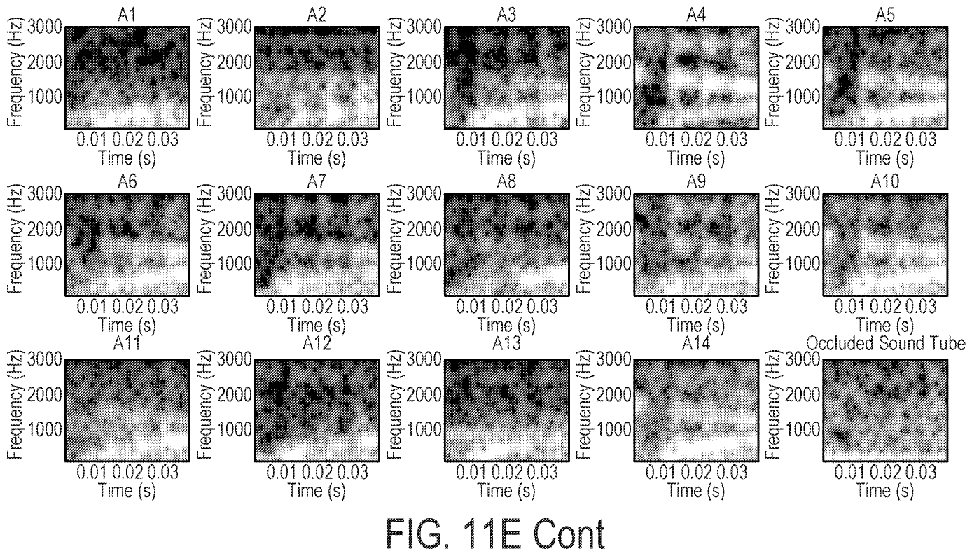

FIGS. 11A-11E illustrate an application of the disclosed methods to obtain neural recording of speech stimuli in cochlear implant patients (i.e., electrical instead of acoustic hearing), which has heretofore not been achievable. In electrical hearing, speech stimuli are delivered through a cochlear implant as a series pulses presented at electrodes and stimulation levels that vary over time (see FIGS. 11A-11C). However, the neural responses to speech stimuli are not able to be identified with the artifact removal methods described in the prior art. Studies in acoustic hearing have shown that the auditory nerve's ability to encode speech stimuli is a strong predictor of word recognition scores (see FIGS. 11D-11E). The invention herein described will facilitate characterization of the auditory nerve's ability to encode speech stimuli in electric hearing.

### C. Conclusion to Removing Stimulation Artifacts from Physiological Recordings

Non-limiting advantages of the disclosed embodiments include: 1) the disclosed methods work for single-pulse and pulse-train stimulation; 2) the disclosed methods open a whole new area of research/innovation for precision medicine in cochlear implants; 3) the disclosed methods do not rely on assumptions that are rarely met (as compared to conventional methods of determining neural response): 4) the disclosed methods provide a time savings of 2-4× in recording time because they require fewer recording traces: 5) the disclosed methods can stimulate at higher stimulation levels, which results in cleaner recordings (i.e., better signal-to-noise ratios), because they do not require a higher masker pulse or anodic leading stimulus (which is perceived as louder at equal stimulation level as cathodic leading stimulus).

Non-limiting applications of the disclosed embodiments include: 1) enhance the clinical use of eCAPs (faster data collection time by 2-4×, reliable recordings so doesn't require expert researcher to determine if valid or not); 2) the disclosed methods enable the recording of neural responses to speech stimuli. Therefore, we will be able to understand scientifically how the auditory nerve encodes speech and make changes in cochlear implant device settings for individual patients based on how their nerve responses to speech stimuli (i.e., precision medicine).

While the embodiments of this invention as described herein were developed for cochlear implants, embodiments also apply to any other implantable stimulation device that delivers electrical stimuli and are considered within the scope of this disclosure. Examples include: pacemakers; defibrillators; retinal stimulators; muscle stimulators; spinal cord stimulators; deep brain stimulators; and neural stimulators to treat urinary incontinence, sleep apnea, shoulder subluxation, and the like.

### I. Computing Environment

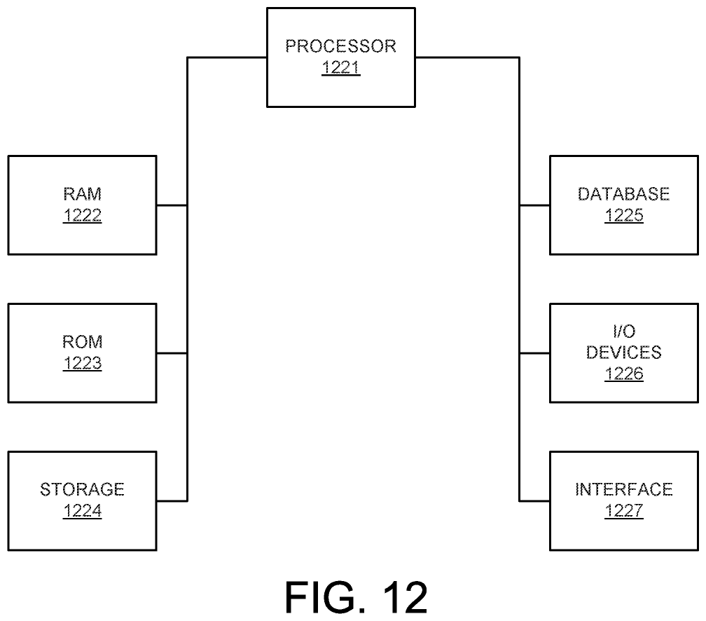

The above-described methods may be implemented on a computing system. The system has been described above as comprised of units. One skilled in the art will appreciate that this is a functional description and that the respective functions can be performed by software, hardware, or a combination of software and hardware. A unit can be software, hardware, or a combination of software and hardware. The units can comprise software for methods of removing stimulation artifacts from physiological recordings after single and multi-pulses. In one exemplary aspect, the units can comprise a computing device that comprises a processor 1221 as illustrated in FIG. 12 and described below.

FIG. 12 illustrates an exemplary computer that can be used for removing stimulation artifacts from physiological recordings after single and multi-pulses. As used herein, “computer” may include a plurality of computers. The computers may include one or more hardware components such as, for example, a processor 1221, a random-access memory (RAM) module 1222, a read-only memory (ROM) module 1223, a storage 1224, a database 1225, one or more input/output (I/O) devices 1226, and an interface 1227. All of the hardware components listed above may not be necessary to practice the methods described herein. Alternatively and/or additionally, the computer may include one or more software components such as, for example, a computer-readable medium including computer executable instructions for performing a method associated with the exemplary embodiments. It is contemplated that one or more of the hardware components listed above may be implemented using software. For example, storage 1224 may include a software partition associated with one or more other hardware components. It is understood that the components listed above are exemplary only and not intended to be limiting.

Processor 1221 may include one or more processors, each configured to execute instructions and process data to perform one or more functions associated with removing stimulation artifacts from physiological recordings after single and multi-pulses. Processor 1221 may be communicatively coupled to RAM 1222, ROM 1223, storage 1224, database 1225, I/O devices 1226, and interface 1227. Processor 1221 may be configured to execute sequences of computer program instructions to perform various processes. The computer program instructions may be loaded into RAM 1222 for execution by processor 1221.

RAM 1222 and ROM 1223 may each include one or more devices for storing information associated with operation of processor 1221. For example, ROM 1223 may include a memory device configured to access and store information associated with the computer, including information for identifying, initializing, and monitoring the operation of one or more components and subsystems. RAM 1222 may include a memory device for storing data associated with one or more operations of processor 1221. For example, ROM 1223 may load instructions into RAM 1222 for execution by processor 1221.

Storage 1224 may include any type of mass storage device configured to store information that processor 1221 may need to perform processes consistent with the disclosed embodiments. For example, storage 1224 may include one or more magnetic and/or optical disk devices, such as hard drives, CD-ROMs, DVD-ROMs, or any other type of mass media device.

Database 1225 may include one or more software and/or hardware components that cooperate to store, organize, sort, filter, and/or arrange data used by the computer and/or processor 1221. For example, database 1225 may store raw data, as described herein and computer-executable instructions for removing stimulation artifacts from physiological recordings after single and multi-pulses. It is contemplated that database 1225 may store additional and/or different information than that listed above.

I/O devices 1226 may include one or more components configured to communicate information with a user associated with computer. For example, I/O devices may include a console with an integrated keyboard and mouse to allow a user to maintain a database of digital images, results of the analysis of the digital images, metrics, and the like. I/O devices 1226 may also include a display including a graphical user interface (GUI) for outputting information on a monitor. I/O devices 1226 may also include peripheral devices such as, for example, a printer for printing information associated with the computer, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 1227 may include one or more components configured to transmit and receive data via a communication network, such as the Internet, a local area network, a workstation peer-to-peer network, a direct link network, a wireless network, or any other suitable communication platform. For example, interface 1227 may include one or more modulators, demodulators, multiplexers, demultiplexers, network communication devices, wireless devices, antennas, modems, and any other type of device configured to enable data communication via a communication network.

### II. Conclusion

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain.

The publications incorporated by reference include, but are not limited to, the following:
A. He, S., Teagle, H. F., & Buchman, C. A. (2017). The electrically evoked compound action potential: from laboratory to clinic. Frontiers in neuroscience, 11, 339.
B. Riggs, W. J., Hiss, M. M., Skidmore, J., Varadarajan, V. V., Mattingly, J. K., Moberly, A. C., & Adunka, O. F. (2020). Utilizing Electrocochleography as a Microphone for Fully Implantable Cochlear Implants. Scientific Reports, 10(1), 1-12.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method of removing stimulation artifacts from physiological recordings to determine a neural response comprising:
receive a recorded waveform trace, wherein the recorded waveform trace includes a stimulation artifact and a neural response, said recorded waveform trace recorded for a first period of time;
model the stimulation artifact of the recorded waveform trace as a rational function by fitting the rational function to the recorded waveform trace; and
subtract the fit rational function from the recorded waveform trace to arrive at the neural response.

2. The method of claim 1, wherein the rational function is a hyperbola.

3. (canceled)

4. (canceled)

5. The method of claim 1, wherein fitting the rational function to the recorded waveform trace comprises fitting the rational function to a first portion of the recorded waveform trace and a second portion of the recorded waveform.

6. (canceled)

7. (canceled)

8. (canceled)

9. The method of claim 1, wherein the patient comprises a patient with a cochlear implant, said method further comprising:
providing trace Bₙ comprising a plurality of pulses to one or more electrodes of the cochlear implant, said plurality of pulses comprising a series of masker pulses prior to application of a probe pulse;
recording trace Cₙ from one or more electrodes of the cochlear implant, where Cₙ includes masker artifacts and neural responses responsive to the masker pulses of trace Bₙ; and
subtracting recorded trace Cₙ from trace Bₙ in order to get just a stimulation artifact and a neural response (Bn-Cn) caused by the probe pulse, wherein the recorded waveform trace comprises the stimulation artifact and a neural response (Bn-Cn) caused by the probe pulse.

10. The method of claim 1, wherein the recorded waveform trace is received from a patient with a cochlear implant, and wherein the neural response comprises an electrically-evoked compound action potential (eCAP).

11. The method of claim 10, wherein the neural response (eCAP) is used to create a neural recording of speech stimuli in cochlear implant patients.

12. A system for removing stimulation artifacts from physiological recordings to determine a neural response (eCAP) comprising:
a memory; and
a processor in communication with the memory, wherein the processor executes computer-executable instructions stored in the memory, said instructions causing the processor to:
receive a recorded waveform trace, wherein the recorded waveform trace includes a stimulation artifact and a neural response, said recorded waveform trace recorded for a first period of time;
model the stimulation artifact of the recorded waveform trace by fitting the rational function to the recorded waveform trace; and
subtract the modeled and fit rational function from the recorded waveform trace to arrive at the neural response (eCAP).

13. The system of claim 12, wherein the rational function is a hyperbola.

14. (canceled)

15. (canceled)

16. The system of claim 12, wherein fitting the rational function to the recorded waveform trace comprises fitting the rational function is fit to a first portion of the recorded waveform trace and a second portion of the recorded waveform.

17. (canceled)

18. (canceled)

19. (canceled)

20. The system of claim 12, wherein the patient comprises a patient with a cochlear implant, wherein the processor executes further computer-executable instructions stored in the memory, said further instructions causing the processor to:
provide trace Bₙ comprising a plurality of pulses to one or more electrodes of the cochlear implant, said plurality of pulses comprising a series of masker pulses prior to application of a probe pulse;
record trace Cₙ from one or more electrodes of the cochlear implant, where Cₙ includes masker artifacts and neural responses responsive to the masker pulses of trace Bₙ; and
subtract recorded trace Cₙ from trace Bₙ in order to get just a stimulation artifact and a neural response (Bₙ-Cₙ) caused by the probe pulse, wherein the recorded waveform trace comprises the stimulation artifact and a neural response (Bₙ-Cₙ) caused by the probe pulse.

21. The system of claim 12, wherein the recorded waveform trace is received from a patient with a cochlear implant, and wherein the neural response comprises an electrically-evoked compound action potential (eCAP).

22. The system of claim 21, wherein the neural response (eCAP) is used to create a neural recording of speech stimuli in cochlear implant patients.

23. A computer-program product comprising computer-executable instructions stored on a non-transitory medium, said computer-executable instructions for performing a method of removing stimulation artifacts from physiological recordings to determine a neural response (eCAP), said method comprising:
receiving a recorded waveform trace, wherein the recorded waveform trace includes a stimulation artifact and a neural response, said recorded waveform trace recorded for a first period of time;
modeling the stimulation artifact of the recorded waveform trace by fitting the rational function to the recorded waveform trace; and
subtracting the modeled and fit rational function from the recorded waveform trace to arrive at the neural response (eCAP).

24. The computer-program product of claim 23, wherein the rational function is a hyperbola.

25. (canceled)

26. (canceled)

27. The computer-program product of claim 23, wherein fitting the rational function to the recorded waveform trace comprises fitting the rational function is fit to a first portion of the recorded waveform trace and a second portion of the recorded waveform.

28. (canceled)

29. (canceled)

30. (canceled)

31. The computer-program product of claim 23, wherein the patient comprises a patient with a cochlear implant, said method further comprising:
providing trace Bₙ comprising a plurality of pulses to one or more electrodes of the cochlear implant, said plurality of pulses comprising a series of masker pulses prior to application of a probe pulse;
recording trace Cₙ from one or more electrodes of the cochlear implant, where Cₙ includes masker artifacts and neural responses responsive to the masker pulses of trace Bₙ;
subtracting recorded trace Cₙ from trace Bₙ in order to get just a stimulation artifact and a neural response (Bₙ-Cₙ) caused by the probe pulse, wherein the recorded waveform trace comprises the stimulation artifact and a neural response (Bₙ-Cₙ) caused by the probe pulse.

32. The computer-program product of claim 23, wherein the recorded waveform trace is received from a patient with a cochlear implant, and wherein the neural response comprises an electrically-evoked compound action potential (eCAP).

33. The computer-program product of claim 32, wherein the neural response (eCAP) is used to create a neural recording of speech stimuli in cochlear implant patients.

34. A method of removing stimulation artifacts from physiological recordings to determine a neural response comprising:
receiving a recorded waveform trace, wherein the recorded waveform trace includes a stimulation artifact and a neural response, said recorded waveform trace recorded for a first period of time;
modeling the stimulation artifact of the recorded waveform trace by fitting a model to the recorded waveform trace; and
subtracting the modeled artifact from the recorded waveform trace to arrive at the neural response.

35. (canceled)

36. (canceled)

37. The method of claim 34, wherein modeling the stimulation artifact of the recorded waveform trace comprises modeling the stimulation artifact as a rational function by fitting the rational function to the recorded waveform trace.

38. The method of claim 37, wherein the rational function is a hyperbola.

39. The method of claim 37, wherein fitting the rational function to the recorded waveform trace comprises fitting the rational function to a first portion of the recorded waveform trace and a second portion of the recorded waveform.

40. (canceled)

41. (canceled)

42. (canceled)

43. The method of claim 34, wherein the patient comprises a patient with a cochlear implant, said method further comprising:
providing trace Bₙ comprising a plurality of pulses to one or more electrodes of the cochlear implant, said plurality of pulses comprising a series of masker pulses prior to application of a probe pulse;
recording trace Cₙ from one or more electrodes of the cochlear implant, where Cₙ includes masker artifacts and neural responses responsive to the masker pulses of trace Bₙ; and
subtracting recorded trace Cₙ from trace Bₙ in order to get just a stimulation artifact and a neural response (Bn-Cn) caused by the probe pulse, wherein the recorded waveform trace comprises the stimulation artifact and a neural response (Bn-Cn) caused by the probe pulse.

44. The method of claim 34, wherein the recorded waveform trace is received from a patient with a cochlear implant, and wherein the neural response comprises an electrically-evoked compound action potential (eCAP).

45. The method of claim 44, wherein the neural response (eCAP) is used to create a neural recording of speech stimuli in cochlear implant patients.
